(19) United States
(12) Patent Application Publication
(10) Pub. No.: US 2015/0335495 A1
(43) Pub. Date: Nov. 26, 2015

(54) Suppoze Rectal Leakage Prevention Device

(71) Applicant: Beth Wigder, Brick, NJ (US)

(72) Inventor: Beth Wigder, Brick, NJ (US)

(21) Appl. No.: 14/722,136

(22) Filed: May 26, 2015

Related U.S. Application Data
(60) Provisional application No. 62/002,663, filed on May 23, 2014.

(51) Int. Cl. A61F 13/20; A61F 13/34; A61F 13/28

(52) U.S. Cl. CPC A61F 13/2011; A61F 13/28; A61F 13/2031; A61F 13/204; A61F 13/2022; A61F 13/206; A61F 13/2068; A61F 13/34; A61F 2013/1513

(57) ABSTRACT

A sanitary, absorbent, disposable insert device that prevents rectal and anal leakage from individuals with a group of illnesses collectively referred to as Inflammatory Bowel Disease, comprising a rolled, multi layered conical shape of absorbent sanitary paper tapering slightly outward towards its lower or distal end, having a pull string at its distal end.

### CLAIM OF PRIORITY

This patent application claims priority under 35 USC 119 (e) (1) from U.S. Provisional Patent Application Ser. No. 62/002,663 filed May 23, 2014, of common inventorship herewith entitled, “The Suppoze,” which is incorporated herein by reference as though the same were set forth in its entirety.

### FIELD OF THE INVENTION

The present invention pertains to the field of hygiene products, and more specifically to the field of rectal leakage aids.

### BACKGROUND OF THE INVENTION

The prior art has put forth several designs for rectal leakage aids. Among these are:

U.S. Pat. No. 5,800,338 to Erek Ethelfeld and Ib Kollerub describes a tampon or closure device for insertion into an external opening of an artificial or natural body canal of an animal or human being. This device includes a resilient compressed plug like body of a molded material, the body having a longitudinal direction and being expandable to a cross sectional dimension ensuring closure of the body canal when inserted in the opening with the longitudinal direction extending longitudinally in the canal. The body is provided with a withdrawal handle element protruding from the body and connected with an anchor part encapsulated in the body and having a relatively large bearing face against the surrounding molded material of the body. The handle element and anchor part are made from a material with a knitted structure to provide a three dimensional bond to the molded material of the body where the anchor part is designed as a soft flexible element oriented in the longitudinal direction.

U.S. Pat. No. 4,979,947 to Irwin R. Berman describes a continence device in the form of a cube or block of resilient foam material capable of being collapsed into a small volume condition and encapsulated in a capsule of gelatin or the like and which includes a string or cord passing through the material and connected with a traction bead engaged with a distal surface of the block or cube or foam material. The continence device provides for voluntary trapping or blockage of gastrointestinal effluent when voluntary and natural control is lacking such as when a person has had a colostomy, ileostomy or in other situations involving fecal incontinence. An introducer is provided for inserting the capsule, the reduced volume soft foam material therein and the line or cord and traction bead into a body orifice such as a stomal orifice, anus of the like. The introducer is in the form of a tubular member, such as a straw which is passed over the cord or string and has one end engaged with the capsule and is provided with a flexible section to facilitate insertion of the device into the desired body orifice with the gelatin capsule then being dissolved in the presence of body warmth and moisture to enable the compressed soft foam material to expand to a predetermined size and shape for blockage and temporary retention of gastrointestinal content in the body orifice. Traction on the string or cord during withdrawal results in compression of the contained foam cube by means of the traction bead facilitating comfortable withdrawal of the continence device from the body orifice.

U.S. Pat. No. 1,561,020 to Edmund E. Pond describes a tampon adapted for medication of the rectal regions. The tampon comprises a shell of soluble material, a body of medicated material supported by said shell and occupying the entire lower portion thereof when it is in operative position and a body of fibrous material supported in said shell above said medication. A protective pad of dry absorbent material is connected to the tampon proper at a distance which locates it outside the anus when the tampon is in operative position. The tampon is inserted in the rectum, the tampon has a cord depending therefrom and a cylinder of medicated material supported by said cord exteriorly of said tampon, whereby when said tampon is in operative position said cylinder will be located in the anus. A tampon adapted for insertion in the rectum, which tampon has a cord depending therefrom and a cylinder of medicated material supported by said cord exteriorly of said tampon to which tampon has a soluble shell, and fibrous packing located in said shell, which packing contains materials adapted, on mixture with water, to generate a small amount of gas, whereby said packing is expanded to more completely fill the cavity in which it is located.

None of these prior art references describe the present invention.

### SUMMARY OF THE INVENTION

It is an object of the present invention to provide a sanitary, absorbent, disposable insert device that prevents rectal and anal leakage from individuals with a group of illnesses collectively referred to as Inflammatory Bowel Disease.

### BRIEF DESCRIPTION OF THE DRAWINGS

FIG. 1 is an illustrative prototypical view showing proximally located indention for medication application, an externally lubricated dome for easy insertion and a distally located pull chord.

FIG. 2 is an illustrative prototypical view showing a distally located shield material for sanitary functionality.

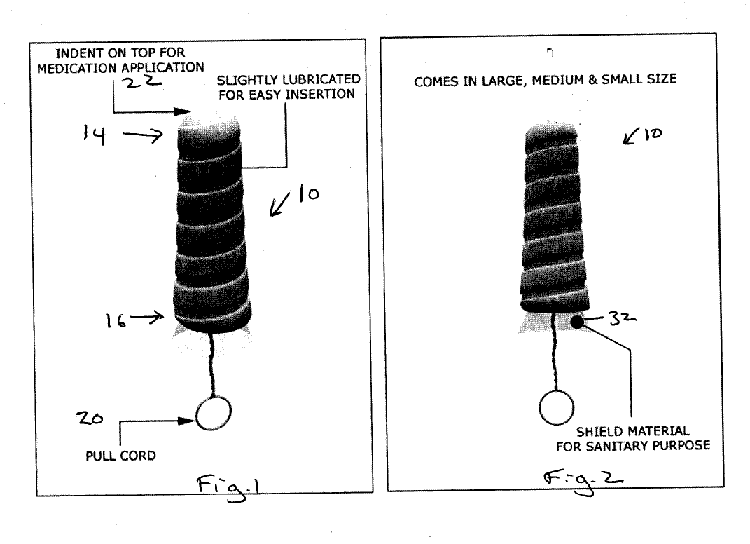

### DETAILED DESCRIPTION OF THE INVENTION

Inventions are always a product of vision and creativity whether they are world changing inventions like cars and computers or everyday conveniences like pump hairsprays, cordless tools and resealable soda bottles. New inventions like a cell phone change a person's life style, making communication more convenient and accessible virtually anywhere. Other inventions such as a snow scraper and brush combine two already existing inventions into an improved embodiment, reducing clutter in our lives and making a daily winter chores much easier. Often necessity combined with a little creativity is the underlying motivator for an invention. Most inventions fill a relatively small need or perform a specialized limited function. If that need is shared by a sufficient number of consumers, an invention has a great potential for success.

A common disease from which many individuals suffer is Inflammatory Bowel Disease or IBD which encompasses many gastro intestinal issues. The two most common IBDs are ulcerative colitis and Crohn's disease. With these illnesses, the immune system mistakes food, bacteria, and other materials in the intestine for foreign substances and attacks the cells of one's intestines. One's body system then sends white blood cells into the lining of the intestines where they produce chronic inflammation. This inflammation results in a host of unpleasant symptoms and consequences from intestinal blockages to chronic diarrhea and rectal bleeding. The afflicted individual often remains housebound, with their existence dramatically restricted by the unpredictable nature of their illness. Another chronic disease that is prevalent among many people is an unrelated condition called Irritable Bowel Syndrome, which renders the afflicted individual susceptible to unpredictable bouts of diarrhea.

The present invention, hereinafter referred to as The Suppoze Rectal Leakage Prevention Device, or simply Suppoze, is a sanitary and disposable insert that prevents anal leakage problems from individuals with a group of illnesses collectively referred to as Inflammatory Bowel Disease. The discreet and effective Suppoze eliminates a need for adult incontinence diapers at an early age and facilitates an active, productive, healthy and normal life free from embarrassment and worry, protected and confident. The Suppoze is an absorbent paper product, slightly conical in shape, analogous to a tampon but manufactured for insertion into one's rectum, for use in protecting an individual from unintended and uncontrollable anal discharges and leakages. The Suppoze has a proximal end, which is smaller diameter than the distal end.

The Suppoze is approximately one and one quarter inches in length. The present invention is produced in small, medium and large diameter sizes. The small size measures approximately three eighths of an inch in diameter, the medium size measures approximately one half inch in diameter, and the large size measures approximately five eighths of an inch in diameter. The present invention comprises a rolled, multi layered conical shape insertion device 10 of absorbent sanitary paper tapering slightly from the narrow proximal end 14 outward towards its lower or distal end 16. One embodiment of The Suppoze is pre lubricated with petroleum jelly or a similar personal lubricant. Another embodiment is non lubricated. The distal end of The Suppoze is equipped with a pull string 20 and the proximal or top end of The Suppoze contains an indentation or recess 22 to accommodate medications for insertion. Also at distal end 16 is a skirt of shield material 32 to prevent touching fecal material when inserting or removing the device. Shield material 32 can be tissue paper, or other suitable material for this purpose.

The Suppoze is easy to use. A user inserts a single Suppoze into their anal canal and pushes it past their sphincter muscle. The Suppoze remains in position until removal either by a user manually pulling the string or by a bowel movement automatically expelling it into a commode. The user changes The Suppoze at least four times a day or every six hours to minimize the risk of bacterial contamination or infection. Disposable by flushing down a commode, The Suppoze is safe for any public sewer or private septic system. Produced in multiple units per package, each unit of The Suppoze is encased individually in a sterile, tear to open wrapper.

The Suppoze provides dramatic freedom of movement for individuals with ulcerative colitis, Irritable Bowel Syndrome symptoms, rectal ulcers and bleeding, hemorrhoids, and diarrhea due to intestinal viruses. The Suppoze is accompanied by relevant instructions and warnings including a statement to consult one's physician before initiating use of The Suppoze. Many individuals suffering from these maladies have little recourse but to attempt a normal daily life while wearing incontinence diapers. The Suppoze provides a superior alternative to bulky incontinence diapers.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A sanitary, absorbent, disposable insert device that prevents rectal or anal leakage comprising: a rolled, multi layered conical shape of absorbent sanitary paper tapering slightly outward towards its lower or distal end.

2. The insert device of claim 1, wherein the length of the device is approximately one and one fourth inches in length and is produced in small, medium and large diameter sizes.

3. The insert device of claim 2 wherein the small diameter size measures approximately three eighths of an inch in diameter.

4. The insert device of claim 2 wherein the medium diameter size measures approximately one half inch in diameter.

5. The insert device of claim 2 wherein the large diameter size measures approximately five eighths of an inch in diameter.

6. The insert device of claim 2, further comprising is pre lubricated with petroleum jelly or a similar personal lubricant.

7. The insert device of claim 2, further comprising a pull string mounted to the distal end of the device.

8. The insert device of claim 2, further comprising a skirt of shield material to prevent touching fecal material when inserting or removing the device.

9. The insert device of claim 7, further comprising a skirt of shield material to prevent touching fecal material when inserting or removing the device.

10. The insert device of claim 2, further comprising an indentation or recess to at the proximal end of the device to accommodate medications for insertion.

11. The insert device of claim 7, further comprising an indentation or recess to at the proximal end of the device to accommodate medications for insertion.

12. The insert device of claim 8, further comprising an indentation or recess to at the proximal end of the device to accommodate medications for insertion.

13. The insert device of claim 2, further comprising an indentation or recess to at the proximal end of the device to accommodate medications for insertion.
